(19) United States
(12) Patent Application Publication
(10) Pub. No.: US 2023/0310869 A1
(43) Pub. Date: Oct. 5, 2023

(54) REMOVABLE PHASE CHANGE MATERIAL TO PRODUCE CONFORMABLE INTERFACE FOR RECHARGING AN IMPLANTED NEUROSTIMULATOR

(71) Applicant: Medtronic, Inc., Minneapolis, MN (US)

(72) Inventors: Andrew Thomas Fried, Saint Paul, MN (US); Robert J. Monson, Roseville, MN (US)

(21) Appl. No.: 17/711,916

(22) Filed: Apr. 1, 2022

(51) Int. Cl. A61N 1/378; H02J 50/10; H02J 50/00; C09K 5/06

(52) U.S. Cl. CPC A61N 1/3787; H02J 50/10; H02J 50/005; C09K 5/063

(57) ABSTRACT

A charging device includes a coil configured to wirelessly transfer energy to an implantable medical device. The charging device includes a phase change material. The charging device may include a ground plane. The phase change material is incorporated in the charging device, and a flexibility of the charging device is associated with a state of the phase change material. The ground plane is conformable to one or more surfaces of a target subject. The charging device may include a heat reduction assembly. The heat reduction assembly includes a material disposed in contact with the coil. The phase change material absorbs at least a portion of heat generated by the coil.

### BACKGROUND

The present disclosure is generally related to implantable medical devices and, in particular, to energy transfer devices and systems for implantable medical devices.

Implantable medical devices may provide a therapeutic result (e.g., pain management, neurostimulation, etc.) for a patient. Some implantable medical devices may utilize energy delivered or transferred from an external charging device. Improved techniques for charging an implanted medical device are desired.

### BRIEF SUMMARY

Example aspects of the present disclosure include:

A charging device including: a coil configured to wirelessly transfer energy to an implantable medical device; and a phase change material.

Any of the aspects herein, wherein: the phase change material is incorporated in the charging device; and a flexibility of the charging device is associated with a state of the phase change material.

Any of the aspects herein, wherein the phase change material is disposed in contact with the coil, wherein the phase change material absorbs at least a portion of heat generated by the coil.

Any of the aspects herein, further including: a second coil associated with operations different from the wireless transfer of the energy to the implantable medical device; wherein at least a portion of the second coil is overlapped by the phase change material, a flexible material, or both.

Any of the aspects herein, wherein: the charging device includes at least one flexible portion; and the phase change material is disposed within the at least one flexible portion.

Any of the aspects herein, wherein the phase change material changes between a solid state and a fluid state at a temperature range of about 35 degrees Celsius to about 41 degrees Celsius.

Any of the aspects herein, wherein: the phase change material is encapsulated in a housing, wherein the housing is insertable into the charging device or both; and an external surface of the housing contacts at least one surface of the coil, at least one surface of a target subject, or both.

Any of the aspects herein, further including: a wearable element, wherein the charging device is insertable into a receptacle of the wearable element, coupled to the wearable element, or both, wherein the phase change material is included in at least one of: the charging device; and the wearable element.

Any of the aspects herein, wherein: a first contact area between the charging device and a target subject under a first temperature condition is different from a second contact area between the charging device and the target subject under a second temperature condition; the first temperature condition corresponds to a first state of the phase change material; and the second temperature condition corresponds to a second state of the phase change material.

Any of the aspects herein, further including a heat reduction assembly, where the heat reduction assembly includes: a material disposed in contact with the coil, wherein the material absorbs at least a portion of heat generated by the coil.

Any of the aspects herein, wherein: the heat reduction assembly is insertable into the charging device; and an external surface of the heat reduction assembly contacts at least one surface of the coil.

Any of the aspects herein, wherein at least a portion of the ground plane includes a flexible material.

Any of the aspects herein, wherein at least a portion of the ground plane includes a thermal conductive material.

Any of the aspects herein, further including: a ground plane, wherein the ground plane is conformable to one or more surfaces of a target subject

Any of the aspects herein, wherein: the ground plane includes a first portion configured to contact one or more surfaces of a target subject and a second portion that is distanced away from the first portion; and the ground plane is configured such that heat is transferred away from the first portion toward the second portion.

A system including: an implantable medical device; a charging device including: a coil configured to wirelessly transfer energy to the implantable medical device; and a phase change material; and electronic circuitry configured to control transferring of the energy to the implantable medical device based on a temperature of the charging device.

Any of the aspects herein, wherein: the charging device includes a heat reduction assembly; and controlling the transferring of the energy to the implantable medical device is based on at least one of: a temperature of the phase change material; a temperature of a component within a threshold distance of the phase change material; a temperature of the heat reduction assembly; and a temperature of a component within a threshold distance of the heat reduction assembly.

Any of the aspects herein, wherein a flexibility of the charging device is associated with a state of the phase change material.

Any of the aspects herein, wherein the charging device includes a ground plane conformable to one or more surfaces of a target subject.

A system including: an implantable medical device; a charging device including: a coil configured to wirelessly transfer energy to the implantable medical device; and a phase change material; a control device including: a processor; and a memory storing data thereon that, when processed by the processor, cause the processor to control transferring of the energy to the implantable medical device based on a temperature of the phase change material.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1 illustrates an example of a system according to at least one implementation of the present disclosure.

FIG. 2 illustrates an example of the system according to at least one implementation of the present disclosure.

FIG. 3 illustrates an example of the system according to at least one implementation of the present disclosure.

FIG. 4 is a process flow according to at least one implementation of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some systems, implanted medical devices (e.g., implanted neurostimulators, neurostimulators having a titanium enclosure, etc.) may provide a therapeutic result (e.g., pain management, neurostimulation, etc.) for a patient. Some implantable medical devices may be rechargeable, as the implantable medical devices may include rechargeable power supplies capable of being charged by energy delivered or transferred from an external charging device. Accordingly, for example, the lifetime power utility of a rechargeable device is relatively high relative compared to devices powered by a primary cell (e.g., a single use battery).

Example drawbacks associated with some rechargeable implantable medical devices include a significant burden to patients associated with regular (e.g., daily, periodic, semi-periodic, continuous, etc.) charging of such implantable medical devices. For example, some implantable medical devices may involve charging (recharging) as often as once or twice per day, with each charging session lasting for 1 to 2 hours. For some implantable medical devices, charging sessions may last 8 hours per day or more (e.g., all day long).

Some external charging device implementations involve a user carrying or wearing a charging device (e.g., a puck-shaped wireless charging device, an antenna connected to a cable, etc.) while charging a corresponding implantable medical device. Accordingly, for example, improved efficiency with respect to charging (e.g., charging duration, charging frequency, etc.) are desired. Additionally, some external charging device implementations may include fixation devices (e.g., a belt or strap, a wearable having a holster, a holder, etc.) to maintain a position of the charging device proximal to the implantable medical device (e.g., maintain a position of an external coil thereof proximal to the implantable medical device). Accordingly, for example, fixation devices that provide user comfort while maintaining the position of the charging device and/or components thereof are desired.

Further, some implementations for recharging an implanted medical device (e.g., an implanted neurostimulator) may be very inefficient. For example, charging duration in some charging processes may be equal to or greater than 1 hour. In some cases, as the implant depth of an implantable medical device increases, more power may need to be produced by an external charging device in association with charging the implantable medical device, which may result in an increased amounts of heat generated by the charging device. In some cases, as the size of the charging device is reduced, the heat capacity of the charging device decreases (e.g., amount of thermal energy that the charging device is capable of retaining before reaching a thermal threshold). As such, a charging device producing high levels of power (and having integrated electronics) may be prone to reaching skin temperature limits (e.g., approximately 41 degrees Celsius) in a relatively short period of time (such as less than 10 or 20 minutes) and before sufficient charging of the device has been achieved.

For example, an external coil of some charging devices may produce between 1.2 watts to 1.5 watts of power (or higher) for cases in which the charging devices are mispositioned (e.g., misaligned with respect to the implantable medical device) or when the implantable medical device is implanted relatively deep (e.g., greater than a threshold depth) in the body of a subject. Accordingly, for example, the temperature of some charging devices may reach skin temperature limits of about 41 degrees Celsius in a relatively short amount of time (e.g., about 10 minutes to 20 minutes from the start of a charging session), depending on thermal boundary conditions.

Such conditions associated with charging an implantable medical device in a patient (e.g., quantity and duration of charging sessions, user inconvenience associated with wearing a fixation device associated with holding a charging device, heat dissipated by a charging device in association with a charging session, etc.) may produce a negative impression for the patient. For example, the majority of the patient's interaction with (and awareness of) therapy systems using such charging devices surrounds a need to constantly charge (recharge) the implantable medical device. Given this negative impression, as alternative therapies become available, the likelihood of the patient changing to such alternative therapies increases.

According to example aspects of the present disclosure, a charging device (also referred to herein as a recharging device or a recharger) capable of charging and/or recharging an implantable medical device is described. The implantable medical device may be an implantable neurostimulator (INS) implanted in a subject (e.g., a patient).

In an example aspect, a phase change material is incorporated in the charging device. The phase change material may be removable from the charging device. In an example, the phase change material may be incorporated into the charging device to produce a conformable interface for charging (recharging) an implantable medical device (e.g., an implanted neurostimulator). The phase change material may be a material (e.g., paraffin wax) having a relatively soft consistency. In some aspects, the phase change material may be a powdered material such as micro-encapsulated phase change material housed in an enclosure. The enclosure may be formed of a flexible, non-phase change material. In some example implementations, the phase change material may be a hybrid mixture of phase change materials and other soft polymers, adhesives, or resins. For example, aspects of the present disclosure support a micro-encapsulated phase change material embedded in an epoxy or medical adhesive such as silicone.

In some aspects, the phase change material (and/or combination of phase change materials) may be positioned in the charging device near a heat source (e.g., a recharge coil) of the charging device. For example, the phase change material may be positioned within a threshold distance of the heat source such that, during a charging process, a temporal duration to reach a threshold temperature (e.g., 41 degrees Celsius) satisfies a threshold value. In some cases, the phase change material may be positioned within a threshold distance of the heat source such that the temporal duration to reach the threshold temperature is extended by a threshold amount (e.g., increased by a temporal value, increased by a percentage, etc.) compared to an implementation without the phase change material.

In some examples, incorporating a phase change material in the charging device may include surrounding the heat source (e.g., recharge coil) with the phase change material. For example, aspects of the present disclosure support disposing the phase change material on top, below, or all around the heat source. In some cases, surrounding the heat source (or a portion thereof) with the phase change material may extend the time to reach a threshold temperature (e.g., 41 degrees Celsius) by 3× or more compared to some other charging devices that do not include a phase change material. In an example, the time to reach the threshold temperature (e.g., incorporating the phase change material) may be equal to about 45 to 60 minutes.

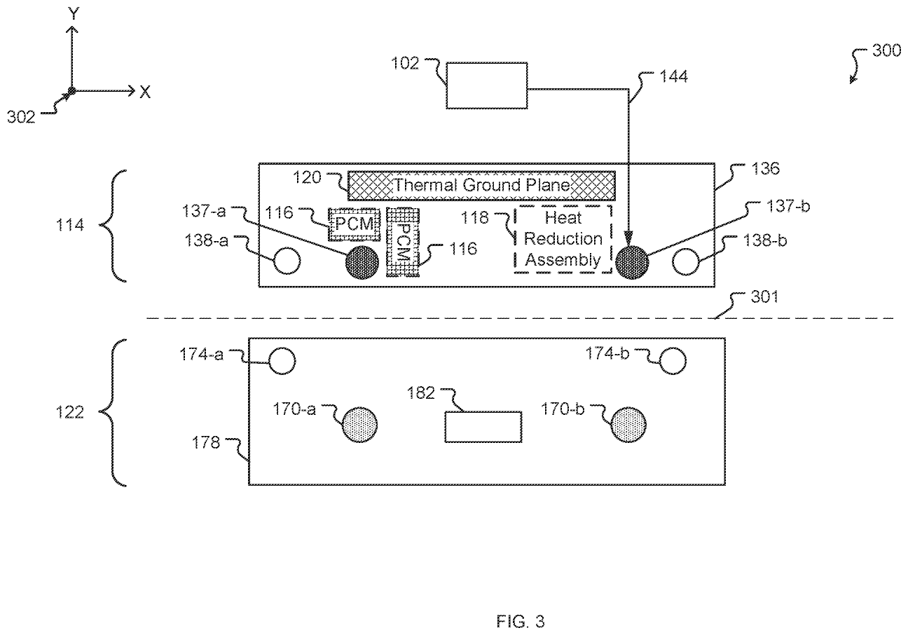

For tibial applications, aspects of the present disclosure support completing an entire charging session (e.g., completely recharging the power supply of the implantable medical device), even when operating at high rates of heat transfer or generated heat (e.g., Qprime, or volumetric heat generation rate) for a majority of the charging session. Qprime may refer to heat in a primary coil (e.g., a primary coil 137 described with reference to FIG. 3) and assembly of the charging device.

Aspects of the present disclosure support a flexible charging antenna (e.g., recharge antenna) of the charging device. For example, the phase change material may be included in a flexible portion of the charging device (e.g., included in the flexible recharge antenna). Aspects of the charging antenna are later described herein.

Properties (e.g., flexibility) of the phase change material may support unhindered flexing of various components (e.g., a recharge coil, the assembly, etc.) of the charging device. In some other aspects, the relatively soft nature of the phase change material may provide increased comfortability to a user (also referred to herein as a patient or subject), as the charging device (e.g., the charging antenna thereof) may be conformable to a surface of the user. For example, during a charging session, the shape of the charging device (e.g., charging antenna) may conform to a body part of the user when the temperature of the phase change material reaches or exceeds a threshold temperature value (e.g., above about 37 degrees Celsius) or a temperature range (e.g., about 37 degrees Celsius to about 40 degrees Celsius, about 35 degrees Celsius to about 41 degrees Celsius). In some aspects, the charging device (e.g., the charging antenna) may retain the shape after cooling back down below the threshold temperature value or below the temperature range. Accordingly, for example, the charging device (e.g., the charging antenna) may retain a shape the patient desires.

Aspects of the present disclosure support the concept of using the phase change material to reduce the heat load transmitted to the patient during a charging session. In an example aspect, an encapsulated phase change material (e.g., a phase change material packet) is insertable into and/or removable from the charging device such that the phase change material is in physical contact with one or more internal components of the charging device. In some aspects, the encapsulated phase change material is in physical contact with a surface (e.g., the skin, clothing, etc.) of the patient.

In some aspects of the present disclosure, a charging system may support user replacement of a phase change material packet (e.g., for which included phase change material has undergone a phase change to a fluid state) with a different packet or pouch (e.g., for which included phase change material is in a solid state). In an example, for patients with a high recharge demand, such features for replacement (e.g., interchangeability) of packets may provide the patients with increased utilization and increased flexibility with respect to using the system. For example, the system may support increased periods of usage before reaching a thermal dose threshold (e.g., in CEM43° C.) for a patient. In some other aspects, the system may support replacement of a phase change material (e.g., a phase change material packet) as the phase change material ages or loses effectiveness due to usage or wear.

In another example aspect, a removable heat sink may be incorporated in the charging device (e.g., within the body of the charging device). The heat sink may absorb heat generated in association with the charging process. In some aspects, the heat sink may be removable from the charging device (e.g., the heat sink may be swappable or interchangeable).

In some aspects, the heat sink (and/or multiple heat sinks) may be positioned in the charging device, adjacent a heat source (e.g., a recharge coil) of the charging device. For example, the heat sink may be positioned in direct contact with the heat source. Additionally, or alternatively, the heat sink may be positioned within a threshold distance of the heat source.

In some aspects, the heat sink may be manufactured of a ceramic material or a composite such as a soft polymer with embedded particles of high heat capacity (e.g., heat capacity equal to or greater than a threshold value). Aspects of the present disclosure support refraining from the use of metallic heat sinks. For example, the use of metal in a heat sink may interfere with the charging efficiency and lead to additional eddy currents. The heat sink may absorb heat generated by the heat source (e.g., in lieu of transmitting the heat to the patient). The heat sink may be cooled (e.g., in a refrigerator or freezer) prior to use, which may increase the amount of time the charging device could be used prior to reaching a thermal dose threshold (e.g., in CEM43° C.) for a patient. In some examples, the charging device may support multiple heat sinks. In some other examples, the system may support multiple instances of inserting (e.g., swapping) a heat sink during a charging session, providing greater flexibility for the patient.

In an example, by incorporating the heat sink, a temporal duration to reach a threshold temperature (e.g., 41 degrees Celsius) during a charging process may satisfy a threshold value. In some cases, by incorporating the heat sink (and/or swapping heat sinks during a charging session) the amount of time the charging device could be used prior to reaching a thermal dose threshold (e.g., in CEM43° C.) may satisfy a threshold.

In some other aspects, the charging device may include a thermal ground plane that is conformable to a surface and/or a body part of the subject. The thermal ground plane may be referred to herein as a conformable thermal ground plane. The thermal ground plane may support the distribution or spreading of heat across the entire contact surface of the thermal ground plane, which may reduce the likelihood of the charging device reaching or exceeding temperature thresholds when charging an implantable medical device (e.g., an INS).

The use of a conformable thermal ground plane at a contact surface (e.g., a side of the charging device in physical contact with a patient) may support heat dissipation. In an example, the base of the charging device may be constructed of a conformable thermal ground plane. Dissipating heat using the conformable thermal ground plane may support a larger amplitude recharge (e.g., relatively higher output power levels by the charging device compared to some systems), which may reduce recharge time associated with a charging (recharging) an implantable medical device. For example, heat generation may be a limiting factor in the recharge cycle in some systems, and incorporation of a conformable thermal ground plane described herein may reduce associated issues described herein arising from the generated heat. Aspects of the present disclosure support keeping relative thicknesses of the conformable thermal ground plane and the heat sink to a minimum (e.g., equal to or less than a threshold thickness) so as to prevent any associated increases in depth from outweighing the benefits described herein of the thermal ground plane and heat sink.

The use of a conformable thermal ground plane as described herein may provide several advantages. In an example aspect, the conformable thermal ground plane may function as an area heat pipe, which may support transferring heat from a portion(s) of the charging device having a relatively higher temperature to a portion(s) of the charging device having a relatively lower temperature. For example, the conformable thermal ground plane may transfer heat away from a hottest point (e.g., of the charging device) in contact with the patient to a coolest point (e.g., of the charging device) in contact with the thermal ground plane. In some aspects, the conformable thermal ground plane may be positioned away from a skin facing side of the charging device. For example, the conformable thermal ground plane may be positioned within the charging device such that the conformable thermal ground plane is located as away from the body of a user as possible. In an example, when the charging device positioned proximal to the implantable medical device in association with a charging session, the conformable thermal ground plane may be relatively furthest from the body in comparison to other internal components of the charging device.

Accordingly, for example, the conformable thermal ground plane may support the spreading (e.g., transfer, dissipation) of any heat generated across the entire contact surface of the thermal ground plane, thereby reducing the likelihood of reaching or exceeding a thermal dose threshold(s) (e.g., in CEM43° C.) for a patient during a charging session. Additionally, the conformable nature of the thermal ground plane would create a more comfortable interface for the patient. In some aspects, the conformable nature of the thermal ground plane may ensure a greater contact area on the patient, thereby increasing the amount of energy that could be transmitted in the form of heat. Accordingly, for example, aspects of the conformable thermal ground plane described herein may support higher recharge energies (e.g., increased transmission power by the charging device), reduced charging time in association with fully charging an implantable medical device (e.g., due to the increased recharge energies), and/or extend available recharge time if necessary (e.g., due to reduced temperature of the charging device). Such features may provide an improved patient experience with respect to charging an implantable medical device (e.g., implanted neurostimulator) implanted in the patient.

Aspects of the present disclosure may be applied to charging systems in which a charging device transfers energy (e.g., charging, recharging) to an implanted medical device. Aspects of the present disclosure may be applied to example implementations in which a surface of the charging device is in physical contact with a subject (and/or in proximity of the subject) during the charging process. Example aspects of the phase change material, the heat sink, and the conformable thermal ground plane are provided herein.

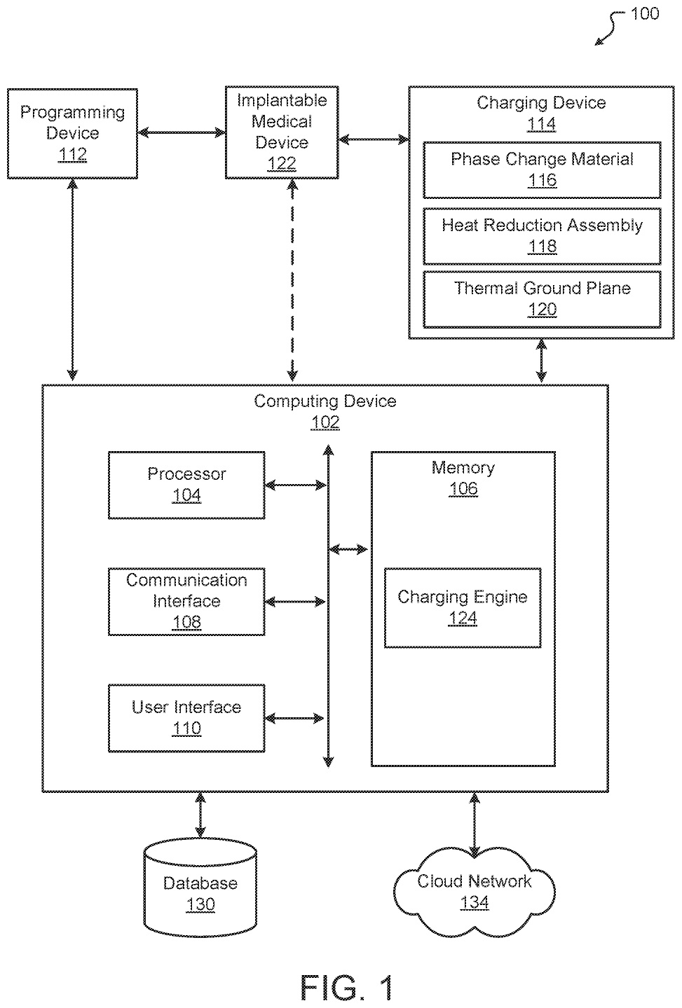

FIG. 1 illustrates an example of a block diagram of a system 100 according to at least one implementation of the present disclosure.

The system 100 includes a computing device 102, a programming device 112, a charging device 114, an implantable medical device 122, a database 130, and/or a cloud network 134 (or other network). Systems according to other implementations of the present disclosure may include more or fewer components than the system 100. For example, the system 100 may omit and/or include additional instances of the programming device 112, the charging device 114, the implantable medical device 122, one or more components of the computing device 102, the database 130, and/or the cloud 134. The system 100 may support the implementation of one or more other aspects of one or more of the methods disclosed herein.

According to example aspects of the present disclosure, the charging device 114 may be capable of charging and/or recharging the implantable medical device 122. The charging device 114 may incorporate any combination of a phase change material 116, a heat reduction assembly 118 (e.g., a heat sink), and a thermal ground plane 120 (e.g., a conformable thermal ground plane) described herein.

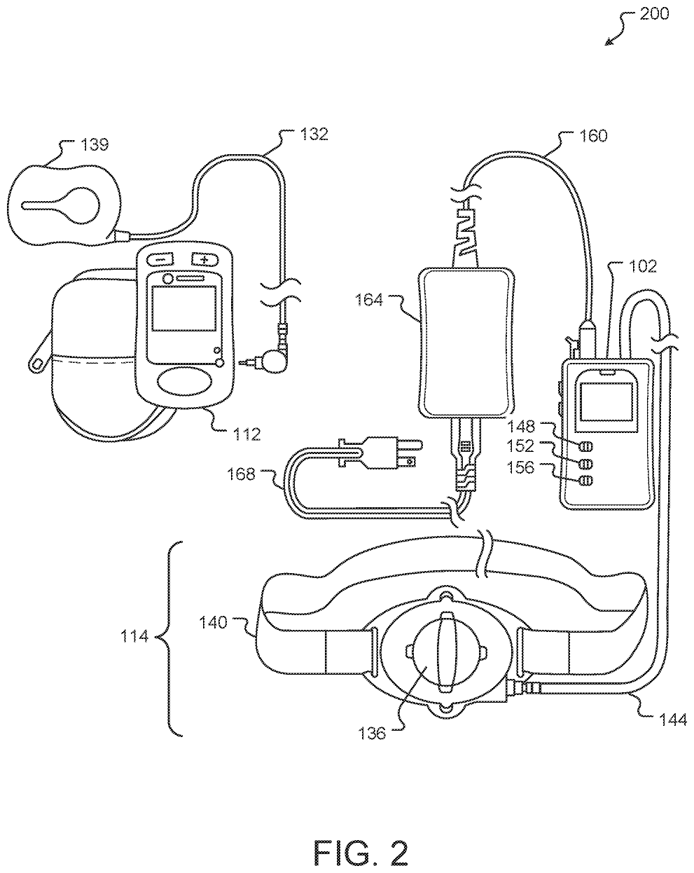

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry associated with driving a charging coil (e.g., a primary coil 137 described with reference to FIG. 3) of the charging device 114. In some aspects, the computing device 102 may be separate from an antenna (e.g., antenna 136 later described with reference to FIG. 2) associated with delivering power to the implantable medical device 122. In some other aspects, the computing device 102 and the antenna (e.g., antenna 136) may be integrated in the same housing. For example, the antenna may be included in the same housing as the computing device 102 and the processor 104.

Non-limiting examples of the computing device 102 may include, for example, personal computing devices or mobile computing devices (e.g., handsets, tablets, wearable devices, mobile phones, smart phones, smart devices, etc.). In some examples, the computing device 102 may be operable by or carried by a human user. In some aspects, the computing device 102 may perform one or more operations autonomously or in combination with an input by the user. The processor 104 of the computing device 102 may be any processor described herein or any similar processor. The processor 104 may be configured to execute instructions stored in the memory 106, which instructions may cause the processor 104 to carry out one or more computing steps utilizing or based on data received from the programming device 112, the charging device 114, the implantable medical device 122, the database 130, and/or the cloud 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the method 400 described herein, or of any other methods. The memory 106 may store, for example, instructions and/or machine learning models that support one or more functions of the programming device 112, the computing device 102, and/or the charging device 114. For instance, the memory 106 may store content (e.g., instructions and/or machine learning models) that, when executed by the processor 104, enable device charging (e.g., by a charging engine 124) and/or a temperature management (e.g., by charging engine 124). Such content, if provided as in instruction, may, in some implementations, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the programming device 112, the charging device 114, the implantable medical device 122, the database 130, and/or the cloud 134. Aspects described herein of the charging engine 124 may be implemented at the computing device 102 and/or the programming device 112.

The computing device 102 may also include a communication interface 108. The communication interface 108 may be used for receiving data or other information from an external source (e.g., the programming device 112, the charging device 114, the implantable medical device 122, the database 130, the cloud 134, and/or any other system or component separate from the system 100), and/or for transmitting instructions, data (e.g., measurements, temperature information, etc.), or other information to an external system or device (e.g., another computing device 102, the programming device 112, the charging device 114, the implantable medical device 122, the database 130, the cloud 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the computing device 102 and one or more other processors 104 or computing devices 102, whether to reduce the time needed to accomplish a computing-intensive task or for any other reason.

In some aspects, the computing device 102 may support direct and indirect communications with any of the programming device 112, the charging device 114, the implantable medical device 122, the database 130, the cloud 134, and/or any other system or component separate from the system 100. In an example, the computing device 102 may communicate directly (e.g., using wireless communications) with the implantable medical device 122. In another example, the computing device 102 may communicate indirectly with the implantable medical device 122 (e.g., via the charging device 114). In some implementations, the computing device 102 may be combined with the charging device 114 in the same assembly.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The programming device 112 may program operations associated with the implantable medical device 122, aspects of which are described with reference to FIG. 2. The programming device 112 may include aspects of the computing device 102 described herein. For example, the programming device 112 may include a processor, a communication interface, memory, content (e.g., executable instructions and/or machine learning models that enable programming a control device), a user interface, or the like described herein.

The charging device 114 (also referred to herein as a recharging device or a recharger) may be capable of charging and/or recharging the implantable medical device 122. Additionally, or alternatively, the charging device 114 may be capable of charging devices (e.g., external medical devices, external neurostimulation devices, etc.) other than the implantable medical device 122. The charging device 114 may include a phase change material 116, a heat reduction assembly 118, and a thermal ground plane 120. In some examples, the charging device 114 may include aspects of the computing device 102. For example, the charging device 114 may also include processors, memory, a communications interface, a user interface, or the like as described with reference to the computing device 102. The charging device 114 may include one or more primary recharge antennas (e.g., a primary coil 137 described with reference to FIG. 3) for transferring power to the implantable medical device 122.

The phase change material 116 may be incorporated into the charging device 114 to produce a conformable interface for charging (recharging) the implantable medical device 122. The phase change material 116 may be a material (e.g., paraffin wax) having a relatively soft consistency. In some implementations, the phase change material 116 may be a liquid (e.g., such as 3M Fluorinert) tunable to change from a liquid phase to vapor phase at around body temperature (e.g., about 37 degrees Celsius). In some aspects, the phase change material 116 may be removable from the charging device 114. For example, the phase change material 116 may be encapsulated in an enclosure (e.g., a phase change material packet, a flexible housing, etc.) that is insertable into and/or removable from the charging device 114. Additionally, or alternatively, the phase change material 116 may be fixed to the charging device 114 (e.g., non-removable).

The heat reduction assembly 118 may be or include, for example, one or more heat sink(s). The heat reduction assembly 118 may be included within the body of the charging device 114. The heat reduction assembly 118 may absorb heat generated in association with operations of the charging device 114 (e.g., heat generated at the charging device 114 in association with charging the implantable medical device 122). In some aspects, the heat reduction assembly 118 may be removable from the charging device 114 (e.g., the heat reduction assembly 118 may be swappable or interchangeable).

The thermal ground plane 120 may be conformable to a surface and/or a body part of the subject. The thermal ground plane 120 may be referred to herein as a conformable thermal ground plane. The thermal ground plane 120 may support the distribution or spreading of heat across an entire contact surface of the thermal ground plane 120, which may reduce the likelihood of the charging device 114 reaching or exceeding temperature thresholds when charging the implantable medical device 122.

In some aspects, for heat generated in association with operations of the charging device 114 (e.g., heat generated at the charging device 114 in association with charging the implantable medical device 122), the thermal ground plane 120 may support the transfer of heat from operating electrical components of the charging device 114 to the heat reduction assembly 118. For example, the thermal ground plane 120 may support transfer of heat to the heat reduction assembly 118 by direct surface contact between the electrical components and the heat reduction assembly 118. Additionally, or alternatively, the thermal ground plane 120 may serve as a medium for transfer of heat from the electrical components and heat sink surfaces.

The thermal ground plane 120 may include compliant or conformable silicone pads, non-silicone based materials (e.g., non-silicone based gap filler materials, thermoplastic and/or thermoset polymeric, elastomeric materials, etc.), silk screened materials, polyurethane foams or gels, thermal putties, thermal greases, thermally-conductive additives, high thermal capacity additives, etc. The thermal ground plane 120 may be configured to have sufficient conformability and/or softness such that the thermal ground plane 120 may closely conform to a mating surface (e.g., flat, non-flat, curved, uneven, etc.) when placed in contact with the mating surface. The thermal ground plane 120 may include a thermally conductive soft thermal interface material formed, for example, from elastomer and at least one thermally-conductive metal, boron nitride, and/or ceramic filler. Accordingly, for example, the thermal ground plane 120 may be conformable without undergoing a phase change or reflow. Additionally, or alternatively, the thermal ground plane 120 may include a thermal interface phase change material.

Additional example aspects of the charging device 114 (e.g., phase change material 116, heat reduction assembly 118, thermal ground plane 120, etc.) are later described herein with reference to FIGS. 2 and 3.

The implantable medical device 122 may be, for example, an implanted neurostimulator (also referred to herein as a neurostimulator device) implantable in a patient. The implantable medical device 122 may deliver neurostimulation therapy to a patient, for example, via one or more leads that include electrodes positioned or located proximate to an anatomical element (e.g., spinal cord, nerves, stomach, brain, etc.) of a patient. The implantable medical device 122 may deliver neurostimulation therapy in the form of electrical pulses. Additional aspects of the implantable medical device 122 are later described herein with reference to FIGS. 2 and 3.

The charging engine 124 may support control of charging operations described herein. For example, the charging engine 124 may control and/or set any combination of parameters (e.g., duration, start time, stop time, scheduling, intensity, etc.) associated with the charging operations. In some aspects, the charging engine 124 may control initiating, pausing, modifying, and/or terminating a charging session.

The charging engine 124 may support monitoring of temperatures of the charging device 114 (e.g., one or more components thereof). For example, the charging engine 124 may monitor or measure temperature values of components (e.g., phase change material 116, heat reduction assembly 118, a heating coil (not illustrated), etc.) of the charging device 114. In some aspects, the charging engine 124 may monitor or measure temperature values at points of contact between the charging device 114 and the patient. In some aspects, the charging engine 124 may control the output of notifications (e.g., haptic, visual, audio, etc.) to the user.

In an example, the charging engine 124 may provide a notification (e.g., via the user interface 110) to the user in association with removing or replacing a component (e.g., phase change material 116, heat reduction assembly 118) of the charging device 114.

For example, the charging engine 124 may monitor temperature information of the charging device 114 (e.g., temperature information of phase change material 116, heat reduction assembly 118, a heating coil (not illustrated), etc.), and the charging engine 124 may provide a notification to remove or replace the phase change material 116 and/or the heat reduction assembly 118 when the temperature information satisfies a set of criteria. For example, the charging engine 124 may generate and provide a notification to remove or replace the phase change material 116 when a temperature value of the charging device 114 (or any component thereof) is greater than or equal to a threshold temperature value. In another example, the charging engine 124 may generate and provide a notification to remove or replace the heat reduction assembly 118 when the temperature value of the charging device 114 (or any component thereof) is greater than or equal to the threshold temperature value.

In some other examples, the charging engine 124 may support replacement of the phase change material 116 (e.g., a phase change material packet) as the phase change material 116 ages or loses effectiveness due to usage. In some cases, the charging engine 124 may support replacement of the heat reduction assembly 118 as the heat reduction assembly 118 ages or loses effectiveness due to usage. For example, the charging engine 124 may analyze temperature performance (e.g., rate of temperature increase) of the charging device 114 in association with a charging session. The charging engine 124 may generate and provide a notification to replace the phase change material 116 and/or the heat reduction assembly 118 when a rate of temperature increase of the charging device 114 (or any component thereof) in association with a charging session is greater than or equal to a threshold value. In some aspects, the charging engine 124 may generate and provide the notification when a rate of temperature increase in association with one or more previous charging sessions is greater than or equal to a threshold value.

In some example implementations, the charging engine 124 may pause a charging session based on the temperature information (e.g., temperature value, rate of temperature increase, etc.) of the charging device 114. In an example, the charging engine 124 may detect when an installed phase change material 116 (and/or heat reduction assembly 118) has been replaced in association with the notification to replace the phase change material 116 (and/or heat reduction assembly 118), and the charging engine 124 may resume the charging session. In some other example implementations, the charging engine 124 may maintain a charging session regardless of the temperature information (e.g., temperature value exceeding a threshold, rate of temperature increase exceeding a threshold, etc.) of the charging device 114. In some examples, the charging engine 124 may maintain a charging session (e.g., refrain from pausing the charging session) even if the phase change material 116 (and/or heat reduction assembly 118) has not been replaced.

The memory 106 (and/or database 130) may store, for example, data associated with one or more charging sessions. For example, the memory 106 (and/or database 130) may include real-time and/or historical temperature information (e.g., temperature values, rates of temperature increase, etc.) described herein of the charging device 114 in association with one or more charging sessions. The memory 106 (and/or database 130) may store temporal information (e.g., date and/or time of installation) and/or identification information associated with a phase change material 116 and/or a heat reduction assembly 118 installed at the charging device 114.

In some aspects, the memory 106 may store machine learning models that support predicting when to replace a phase change material 116 and/or a heat reduction assembly 118 in association with a charging session. For example, the machine learning models may support predicting when the phase change material 116 may reach a target temperature. In another example, the machine learning models may support prediction when the heat reduction assembly 118 may reach a target temperature. In an example, the machine learning models may determine the predictions based on the data associated with the charging sessions.

Accordingly, for example, the charging engine 124 may generate and provide a notification associated with replacing the phase change material 116 and/or the heat reduction assembly 118 based on the predictions. In an example, the notification may include an indication to replace the phase change material 116 and/or the heat reduction assembly 118. Additionally, or alternatively, the notification may include an indication of a remaining time until replacement should be performed. In some other aspects, the memory 106 may store machine learning models that support predicting when to start, pause, modify, and/or end a charging session. For example,

The database 130 may store information that correlates one coordinate system to another. The database 130 may additionally or alternatively store, for example, location or coordinates of the implantable medical device 122). The database 130 may be configured to provide any such information to the computing device 102 or to any other device of the system 100 or external to the system 100, whether directly or via the cloud 134. In some implementations, the database 130 may include treatment information (e.g., a pain management plan) associated with a patient. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of the methods 400 described herein. The system 100 or similar systems may also be used for other purposes.

FIG. 2 illustrates an example 200 of the system 100 of FIG. 1.

The computing device 102 may receive AC power from a power source (not illustrated) via a cable 168 and a transformer 164. In some aspects, the computing device 102 may include rechargeable power source (not illustrated), such as a battery. The computing device 102 may provide power (e.g., via the charging device 114) to the implantable medical device 122 from the power source and/or the rechargeable power source.

The user interface 110 may support controls (not illustrated) associated with starting, pausing, modifying, or stopping a therapy session and/or a charging session (e.g., based on implementation). In some aspects, the user interface 110 may support controls (e.g., a “Start Charge” button 148, a “Stop Charge” button 152, and a “Silence” button 156) associated with starting, pausing, modifying, or stopping a therapy session and/or a charging session. The “Start Charge” button 148 and the “Stop Charge” button 152 may support starting and stopping of a therapy session and/or a charging session. The “Silence” button 156 may support disabling and/or re-enabling of notifications (e.g., video, audio, and/or haptic) by the computing device 102 in association with a therapy session and/or a charging session.

The programming device 112 is attachable via a cable 132 to an external telemetry coil 139. Programming device 112 may program operations of the implantable medical device 122 using the external telemetry coil 139. In some aspects, the programming device 112 may manipulate therapeutic programs such as starting therapy and stopping therapy.

The charging device 114 includes an antenna 136 capable of inductively transferring power to the implantable medical device 122. In some aspects, the antenna 136 may be capable of capacitively transferring power to the implantable medical device 122. The charging device 114 (e.g., antenna 136) is attachable via a cable 144 to computing device 102. The computing device 102 may provide power to the charging device 114 via the cable 144. In some example implementations, the computing device 102 and the charging device 114 may be integrated in the same housing. For example, the antenna 136 may be included in the same housing as the computing device 102.

The charging device 114 (e.g., antenna 136) may be positioned (e.g., held in a position) with respect to a patient using a wearable element 140. In an example, the wearable element 140 may be a belt, a band, a strap, an item of clothing (e.g., a shirt, pants, a jacket, an undergarment, etc.) or the like. In some other aspects, the wearable element 140 may include a receptacle for holding the charging device 114. The wearable element 140 may be adjustable based on one or more dimensions of the user. In some aspects, the wearable element 140 may be manufactured of any combination of fabrics, flexible materials (e.g., rubber, plastic, etc.), elastic materials, and rigid materials. In some aspects, the charging device 114 may be separate from the wearable element 140. In some other aspects, the charging device 114 may be integrated with the wearable element 140.

FIG. 3 illustrates an example 300 of the system 100 of FIG. 1. In example 300, the implantable medical device 122 is located beneath a cutaneous boundary 301 (e.g., the skin) of a patient, and the charging device 114 is positioned in association with charging the implantable medical device 122.

Features of the system 100 may be described in conjunction with a coordinate system 302. The coordinate system 302 includes three-dimensions comprising an X-axis, a Y-axis, and a Z-axis (not illustrated). Additionally or alternatively, the coordinate system 302 may be used to define planes (e.g., the XY-plane, the XZ-plane, and the YZ-plane) of the system 100. These planes may be disposed orthogonal, or at 90 degrees, to one another. While the origin of the coordinate system 302 may be placed at any point on or near the components of the system 100, for the purposes of description, the axes of the coordinate system 302 are always disposed along the same directions from figure to figure, whether the coordinate system 302 is shown or not. In some examples, reference may be made to dimensions, angles, directions, relative positions, and/or movements associated with one or more components of the system 100 with respect to the coordinate system 302.

Charging device 114 includes an antenna 136. Charging device 114 may include primary coils 137 (e.g., primary coil 137-a, primary coil 137-b). In an example, the computing device 102 may include electronic circuitry capable of driving the primary coils 137 with an oscillating current. Charging device 114 includes external telemetry coils 138 (e.g., external telemetry coil 138-a, external telemetry coil 138-b) supportive of wireless communications with other devices (e.g., programming device 112, implantable medical device 122, etc.). In some aspects, the primary coils 137 and/or external telemetry coils 138 may be included in the antenna 136. In some example implementations, the primary coils 137 and the external telemetry coils 138 may be located on the same plane (e.g., XZ-plane) of the charging device 114.

Implantable medical device 122 includes internal telemetry coils 174 (e.g., internal telemetry coil 174-a, internal telemetry coil 174-b) supportive of wireless communications with other devices (e.g., programming device 112, charging device 114, etc.). In an example, the programming device 112 may program and/or control implantable medical device 112 using wireless communications described herein. In some aspects, the programming device 112 may obtain information (e.g., data) from the implantable medical device 122 using wireless communications.

Implantable medical device 122 includes secondary coils 170 (e.g., secondary coil 170-a, secondary coil 170-b) (also referred to herein as secondary charging coils). The secondary coils 170 support charging of the implantable medical device 122. For example, the implantable medical device 122 includes a rechargeable power source 182 that is chargeable when the implantable medical device 122 is in an implanted state (e.g., implanted in a patient).

For example, when a primary coil 137 (e.g., primary coil 137-a) driven with an oscillating current is positioned in proximity of a secondary coil 170 (e.g., secondary coil 170-a) of the implantable medical device 122, the primary coil 137 may induce a current in the secondary coil 170, thereby charging the rechargeable power source 182. For example, the primary coil 137 may induce a current in the secondary coil 170 when the primary coil 137 is in proximity (e.g., within a threshold distance) of the secondary coil 170. In some aspects, a primary coil 137 (e.g., primary coil 137-a, primary coil 137-b) may be positioned or located in the charging device 114 such that the primary coil 137 is relatively proximal to secondary coil 170 (e.g., secondary coil 170-a, secondary coil 170-b) when the charging device 114 is positioned near the implantable medical device 122 in association with a charging session.

In some aspects, computing device 102 may be separate from or integrated with the charging device 114. For example, computing device 102 may be operatively coupled to the antenna 136 (and primary coils 137) of the charging device 114 by a cable 144. Additionally, or alternatively, the computing device 102 and antenna 136 may be combined into a single device. Accordingly, for example, the computing device 102 may support direct and indirect communications with another device (e.g., components of the system 100, components of another system, etc.). In an example, the computing device 102 may communicate directly (e.g., using wireless communications) with the implantable medical device 122. In another example, the computing device 102 may communicate indirectly with the implantable medical device 122 (e.g., via the charging device 114, when separate from the charging device 114).

In some aspects, one or more dimensions (e.g., diameter) of each internal telemetry coil 174 (e.g., internal telemetry coil 174-a) may be larger than a corresponding dimension (e.g., diameter) of a secondary charging coil 170 (e.g., secondary charging coil 170-a).

In some aspects, the secondary charging coils 170 (e.g., secondary charging coil 170-a, secondary charging coil 170-b) may be located in a middle position of the implantable medical device 122 (e.g., with respect to the Y-axis). The positioning of the secondary charging coils 170 may support “flipping” in the body of the implantable medical device 122 in association with deep brain stimulation (DBS) or spinal cord stimulation (SCS) when the implantable medical device 122 is a leaded device (e.g., electrically connected to a device configured to control stimulation). The implantable medical device 122 may communicate (e.g., send and receive data signals) with the programming device 112, computing device 102, or other device using wireless communications described herein.

Example implementations of the charging device 114 may include any combination of the phase change material 116 (illustrated as ‘PCM 116’ in FIG. 3), the heat reduction assembly 118, and the thermal ground plane 120 described herein. For example, the charging device 114 may include or omit any of the phase change material 116, the heat reduction assembly 118, or the thermal ground plane 120. In some example implementations, the phase change material 116 may be located at a top side of the charging device 114 (e.g., with respect to the Y-axis) and/or the middle of the charging device 114 (e.g., with respect to any of the X through Z axes). The phase change material 116 may be in physical contact with a primary coil 137 (or other component of the charging device 114) and support cooling the primary coil 137 (or other component of the charging device 114). In some example implementations, the heat reduction assembly 118 may be located in the middle of the charging device 114 (e.g., with respect to any of the X through Z axes). The heat reduction assembly 118 may be in physical contact with a primary coil 137 (or other component of the charging device 114) and support cooling the primary coil 137 (or other component of the charging device 114). In some example implementations, the thermal ground plane 120 may be located at a relatively higher position (e.g., with respect to the Y-axis) of the charging device 114.

Example aspects of the system 100 supported by the present disclosure are described herein with reference to FIGS. 1 through 3.

A charging device (e.g., charging device 114) includes a coil (e.g., a primary coil 137) configured to wirelessly transfer energy to an implantable medical device (e.g., implantable medical device 122). The charging device includes a phase change material (e.g., phase change material 116). In some alternative and/or additional aspects, the charging device includes a ground plane (e.g., thermal ground plane 120). In some aspects, the ground plane is conformable to one or more surfaces of a target subject.

In some aspects, the phase change material is incorporated in the charging device. In an example, a flexibility of the charging device is associated with a state (e.g., a solid state, a fluid state) of the phase change material.

In some aspects, the phase change material is disposed in contact with the coil (e.g., adjacent the coil, in physical contact with the coil), wherein the phase change material absorbs at least a portion of heat generated by the coil. In some examples, the phase change material is disposed above, below, and/or adjacent the coil. In some cases, the phase change material is in physical contact with at least a portion of the coil. In some aspects, the phase change material reduces a rate of temperature change of the coil in association with transferring the energy to the implantable medical device. In some other aspects, the phase change material reduces a temperature of the coil.

In some aspects, the charging device includes a second coil associated with operations different from the wireless transfer of energy to the implantable medical device. For example, the second coil may be an external telemetry coil 138 described with reference to FIG. 1. In another example, the second coil may support communicating data with the implantable medical device. In an example, at least a portion of the second coil is overlapped by the phase change material.

In some aspects, the charging device includes a second coil (e.g., an external telemetry coil 138) associated with communicating data with the implantable medical device. In some examples, at least a portion of the second coil includes a flexible material.

In some aspects, the charging device comprises at least one flexible portion. In some examples, the phase change material is disposed within the at least one flexible portion.

In some aspects, the phase change material changes between a solid state and a fluid state at a temperature of about 37 degrees Celsius. For example, the phase change material may change between a solid state and a fluid state at a temperature range about 37 degrees Celsius to about 40 degrees Celsius. In another example, the phase change material may change between a solid state and a fluid state at a temperature range of about 35 degrees Celsius to about 41 degrees Celsius.

In some alternative and/or additional aspects, the phase change material may be in the form of micro-encapsulated phase change particles. In an example, each micro-encapsulated phase change particle may be formed of a phase change material (also referred to as a core or fill) described herein, encapsulated by a flexible, non-phase change material (also referred to herein as an enclosure, a shell, a coating, or a membrane). Each micro-encapsulated phase change particle may remain in a particle form, even after a change of phase (e.g., to a fluid state) of the phase change material encapsulated therein. For example, a set of micro-encapsulated phase change particles may remain in powder form, even after a change of phase of the phase change material encapsulated in each particle.

In some aspects, the phase change material is encapsulated in a housing (e.g., a pouch, a packet). In an example, the housing is insertable into the charging device. In some examples, an external surface of the housing contacts at least one surface of the coil, at least one surface of a target subject, or both. In some examples, the housing may be formed of a flexible or semi-flexible material.

In some aspects, the charging device further includes a wearable element (e.g., wearable element 140). In an example, the charging device is insertable into a receptacle of the wearable element, coupled to the wearable element, or both. In some examples, the phase change material is included in at least one of: the charging device; and the wearable element. In some examples, one or more dimensions of the receptacle correspond to one or more dimensions of the charging device.

In some aspects, a first contact area between the charging device and a target subject under a first temperature condition is different from a second contact area between the charging device and the target subject under a second temperature condition. In an example, the first temperature condition corresponds to a first state (e.g., a solid state) of the phase change material, and the second temperature condition corresponds to a second state (e.g., a fluid state) of the phase change material.

In some aspects, the charging device includes a heat reduction assembly (e.g., heat reduction assembly 118). In an example, the heat reduction assembly includes a material (e.g., ceramic, metal, etc.) disposed in contact with the coil (e.g., adjacent the coil, in physical contact with the coil). In an example, the material absorbs at least a portion of heat generated by the coil.

In some aspects, the heat reduction assembly is insertable into the charging device. In an example, an external surface of the heat reduction assembly contacts at least one surface of the coil. In some examples, the external surface of the heat reduction assembly contacts at least one heat generating component of the charging device. In some aspects, the heat reduction assembly may include the phase change material described herein.

In some aspects, at least a portion of the ground plane includes a flexible material.

In some aspects, at least a portion of the ground plane includes a thermal conductive material.

In some aspects, the ground plane includes a first portion configured to contact one or more surfaces of a target subject and a second portion that is distanced away from the first portion. In an example, the ground plane is configured such that heat is transferred away from the first portion toward the second portion. In an example, a temperature of the first portion is different from a temperature of the second portion.

In some aspects, at least one of an effective directionality associated with transferring the energy and an effective power associated with transferring the energy is associated with a state of the phase change material. For example, bending of the charging device (e.g., charging device 114, due to phase change) may provide improved directionality associated with transferring the energy from a primary coil 137 to a secondary charging coil 170 (e.g., provide improved induction of current in the secondary coil 170), which may provide an increase in overall charging efficiency.

A system (e.g., system 100) includes an implantable medical device (e.g., implantable medical device 122), a charging device (e.g., charging device 114), and electronic circuitry (e.g., at the charging device 114, at a programming device 112) configured to control transferring of the energy to the implantable medical device based on a temperature of the phase change material.

The charging device may include a coil (e.g., primary coil 137) configured to wirelessly transfer energy to the implantable medical device. The charging device may include a phase change material (e.g., phase change material 116). In some alternative and/or additional aspects, the charging device may include a ground plane (e.g., thermal ground plane 120). In some examples, the ground plane is conformable to one or more surfaces of a target subject.

In some aspects, the charging device includes a heat reduction assembly (e.g., heat reduction assembly 118).

In some aspects, the system includes a control device (e.g., charging device 114, programming device 112) including a processor and a memory storing data thereon that, when processed by the processor, cause the processor to control transferring of the energy to the implantable medical device based at least in part on a temperature of the charging device. For example, the electronic circuitry is configured to control the transferring of the energy to the implantable medical device based on a temperature of the phase change material. In some other aspects, the data, when processed by the processor, cause the processor to control the transferring of the energy to the implantable medical device based at least in part on a temperature of the heat reduction assembly. In some cases, the electronic circuitry is configured to control the transferring of the energy to the implantable medical device based on a temperature of components located within a threshold distance of (e.g., nearby, adjacent, in contact with) the phase change material or the heat reduction assembly.

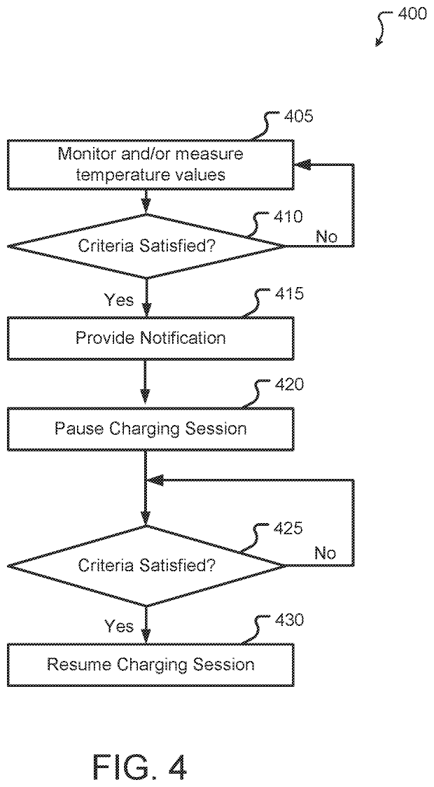

FIG. 4 illustrates an example of a process flow 400 in accordance with aspects of the present disclosure. In some examples, process flow 400 may implement aspects of a computing device 102, programming device 112, and/or charging device 114 described with reference to FIGS. 1 through 3.

In the following description of the process flow 400, the operations may be performed in a different order than the order shown, or the operations may be performed in different orders or at different times. Certain operations may also be left out of the process flow 400, or other operations may be added to the process flow 400.

It is to be understood that any of the operations of process flow 400 may be performed by any device (e.g., a computing device 102, a programming device 112, a charging device 114, etc.).

At 405, the process flow 400 includes monitoring (e.g., by the charging engine 124) and/or measuring temperature values of components (e.g., phase change material 116, heat reduction assembly 118, thermal ground plane 120, a primary coil 137, etc.) of the charging device 114 in association with a charging session.

At 410, the process flow 400 includes identifying whether temperature information of the charging device 114 (e.g., temperature information of phase change material 116, temperature information of the heat reduction assembly 118, temperature information of the primary coil 137, etc.) satisfies one or more criteria.

In an example, the one or more criteria may include a threshold temperature value. In another example, the one or more criteria may include a threshold rate of temperature increase at one or more components of the charging device 114 (e.g., at the phase change material 116, at the heat reduction assembly 118, at the primary coil 137, etc.).

At 415, the process flow 400 includes generating and/or providing (e.g., by the charging engine 124) a notification associated with the charging session, based on the temperature information satisfying one or more of the criteria (e.g., ‘Yes’).

In an example, for a case in which phase change material 116 is installed in the charging device 114, the notification may include an indication to remove and/or replace the phase change material 116. Additionally, or alternatively, for the case in which phase change material 116 is installed in the charging device 114, the notification may include an indication to insert additional phase change material 116. In some other aspects, for a case in which no phase change material 116 is installed in the charging device 114, the notification may include an indication to insert the phase change material 116.

In another example, for a case in which a heat reduction assembly 118 is installed in the charging device 114, the notification may include an indication to remove and/or replace the heat reduction assembly 118. Additionally, or alternatively, for the case in which the heat reduction assembly 118 is installed in the charging device 114, the notification may include an indication to insert an additional heat reduction assembly 118. In some other aspects, for a case in which no heat reduction assembly 118 is installed in the charging device 114, the notification may include an indication to insert the heat reduction assembly 118.

In some other aspects, the notification may include an indication to remove and/or replace any combination of the phase change material 116 and the heat reduction assembly 118. For example, the notification may include an indication to insert any quantity of the phase change material 116 and/or the heat reduction assembly 118.

Additionally, or alternatively, at 420, the process flow 400 includes pausing the charging session. In some aspects, the notification at 415 may include an indication of temporal information associated with pausing the charging session. For example, the temporal information may include a time instance of when the charging session will be paused (e.g., X minutes until the charging session will be paused).

At 425, the process flow 400 includes detecting whether user input associated with resuming the charging session satisfies one or more criteria. For example, the user input may include a user selection of the “Start Button” 148 described with reference to FIG. 2. In another example, the user input may include installation of a phase change material 116 (e.g., a replacement phase change material 116, an additional phase change material 116) and/or a heat reduction assembly 118 (e.g., a replacement heat reduction assembly 118, an additional heat reduction assembly 118).

At 430, based on detecting that a phase change material 116 (and/or heat reduction assembly 118) has been installed in the charging device 114 in association with the notification (e.g., ‘Yes’), the charging engine 124 may resume the charging session.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

A charging device including: a coil configured to wirelessly transfer energy to an implantable medical device; and a phase change material.

Any of the aspects herein, wherein: the phase change material is incorporated in the charging device; and a flexibility of the charging device is associated with a state of the phase change material.

Any of the aspects herein, wherein the phase change material is disposed in contact with the coil, wherein the phase change material absorbs at least a portion of heat generated by the coil.

Any of the aspects herein, further including: a second coil associated with operations different from the wireless transfer of the energy to the implantable medical device; wherein at least a portion of the second coil is overlapped by the phase change material, a flexible material, or both.

Any of the aspects herein, wherein: the charging device includes at least one flexible portion; and the phase change material is disposed within the at least one flexible portion.

Any of the aspects herein, wherein the phase change material changes between a solid state and a fluid state at a temperature range of about 35 degrees Celsius to about 41 degrees Celsius.

Any of the aspects herein, wherein: the phase change material is encapsulated in a housing, wherein the housing is insertable into the charging device or both; and an external surface of the housing contacts at least one surface of the coil, at least one surface of a target subject, or both.

Any of the aspects herein, further including: a wearable element, wherein the charging device is insertable into a receptacle of the wearable element, coupled to the wearable element, or both, wherein the phase change material is included in at least one of: the charging device; and the wearable element.

Any of the aspects herein, wherein: a first contact area between the charging device and a target subject under a first temperature condition is different from a second contact area between the charging device and the target subject under a second temperature condition; the first temperature condition corresponds to a first state of the phase change material; and the second temperature condition corresponds to a second state of the phase change material.

Any of the aspects herein, further including a heat reduction assembly, where the heat reduction assembly includes: a material disposed in contact with the coil, wherein the material absorbs at least a portion of heat generated by the coil.

Any of the aspects herein, wherein: the heat reduction assembly is insertable into the charging device; and an external surface of the heat reduction assembly contacts at least one surface of the coil.

Any of the aspects herein, wherein at least a portion of the ground plane includes a flexible material.

Any of the aspects herein, wherein at least a portion of the ground plane includes a thermal conductive material.

Any of the aspects herein, further including: a ground plane, wherein the ground plane is conformable to one or more surfaces of a target subject

Any of the aspects herein, wherein: the ground plane includes a first portion configured to contact one or more surfaces of a target subject and a second portion that is distanced away from the first portion; and the ground plane is configured such that heat is transferred away from the first portion toward the second portion.

A system including: an implantable medical device; a charging device including: a coil configured to wirelessly transfer energy to the implantable medical device; and a phase change material; and electronic circuitry configured to control transferring of the energy to the implantable medical device based on a temperature of the charging device.

Any of the aspects herein, wherein: the charging device includes a heat reduction assembly; and controlling the transferring of the energy to the implantable medical device is based on at least one of: a temperature of the phase change material; a temperature of a component within a threshold distance of the phase change material; a temperature of the heat reduction assembly; and a temperature of a component within a threshold distance of the heat reduction assembly.

Any of the aspects herein, wherein a flexibility of the charging device is associated with a state of the phase change material.

Any of the aspects herein, wherein the charging device includes a ground plane conformable to one or more surfaces of a target subject.

A system including: an implantable medical device; a charging device including: a coil configured to wirelessly transfer energy to the implantable medical device; and a phase change material; a control device including: a processor; and a memory storing data thereon that, when processed by the processor, cause the processor to control transferring of the energy to the implantable medical device based on a temperature of the phase change material.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A charging device comprising:
a coil configured to wirelessly transfer energy to an implantable medical device; and
a phase change material.

2. The charging device of claim 1, wherein:
the phase change material is incorporated in the charging device; and
a flexibility of the charging device is associated with a state of the phase change material.

3. The charging device of claim 1, wherein:
the phase change material is disposed in contact with the coil, wherein the phase change material absorbs at least a portion of heat generated by the coil.

4. The charging device of claim 1, further comprising:
a second coil associated with operations different from the wireless transfer of the energy to the implantable medical device,
wherein at least a portion of the second coil is overlapped by the phase change material, a flexible material, or both.

5. The charging device of claim 1, wherein:
the charging device comprises at least one flexible portion; and
the phase change material is disposed within the at least one flexible portion.

6. The charging device of claim 1, wherein the phase change material changes between a solid state and a fluid state at a temperature range of about 35 degrees Celsius to about 41 degrees Celsius.

7. The charging device of claim 1, wherein:
the phase change material is encapsulated in a housing, wherein the housing is insertable into the charging device or both; and
an external surface of the housing contacts at least one surface of the coil, at least one surface of a target subject, or both.

8. The charging device of claim 1, further comprising:
a wearable element, wherein the charging device is insertable into a receptacle of the wearable element, coupled to the wearable element, or both,
wherein the phase change material is comprised in at least one of:
the charging device; and
the wearable element.

9. The charging device of claim 1, wherein:
a first contact area between the charging device and a target subject under a first temperature condition is different from a second contact area between the charging device and the target subject under a second temperature condition;
the first temperature condition corresponds to a first state of the phase change material; and
the second temperature condition corresponds to a second state of the phase change material.

10. The charging device of claim 1, further comprising a heat reduction assembly, wherein the heat reduction assembly comprises:
a material disposed in contact with the coil,
wherein the material absorbs at least a portion of heat generated by the coil.

11. The charging device of claim 1, wherein:
the heat reduction assembly is insertable into the charging device; and
an external surface of the heat reduction assembly contacts at least one surface of the coil.

12. The charging device of claim 1, wherein at least a portion of the ground plane comprises a flexible material.

13. The charging device of claim 1, wherein at least a portion of the ground plane comprises a thermal conductive material.

14. The charging device of claim 1, further comprising:
a ground plane, wherein the ground plane is conformable to one or more surfaces of a target subject

15. The charging device of claim 14, wherein:
the ground plane comprises a first portion configured to contact one or more surfaces of a target subject and a second portion that is distanced away from the first portion; and
the ground plane is configured such that heat is transferred away from the first portion toward the second portion.

16. A system comprising:
an implantable medical device;
a charging device comprising:
a coil configured to wirelessly transfer energy to the implantable medical device; and
a phase change material; and
electronic circuitry configured to control transferring of the energy to the implantable medical device based at least in part on a temperature of the charging device.

17. The system of claim 16, wherein:
the charging device comprises a heat reduction assembly; and
controlling the transferring of the energy to the implantable medical device is based at least in part on at least one of:
a temperature of the phase change material;
a temperature of a component within a threshold distance of the phase change material;
a temperature of the heat reduction assembly; and
a temperature of a component within a threshold distance of the heat reduction assembly.

18. The system of claim 17, wherein a flexibility of the charging device is associated with a state of the phase change material.

19. The system of claim 16, wherein:
the charging device comprises a ground plane conformable to one or more surfaces of a target subject.

20. A system comprising:
an implantable medical device;
a charging device comprising:
a coil configured to wirelessly transfer energy to the implantable medical device; and
a phase change material;
a control device comprising:
a processor; and
a memory storing data thereon that, when processed by the processor, cause the processor to control transferring of the energy to the implantable medical device based at least in part on a temperature of the phase change material.
